(19) United States
(12) Patent Application Publication
(10) Pub. No.: US 2016/0176890 A1
(43) Pub. Date: Jun. 23, 2016

(54) Oral Pharmaceutical Compositions of Buprenorphine and Another Opioid Receptor Agonist

(71) Applicant: Relmada Therapeutics, Inc., New York, NY (US)

(72) Inventors: Najib BABUL, Blue Bell, PA (US); Ashish Kumar Rehni, Patiala (IN)

(73) Assignee: Relmada Therapeutics, Inc., New York, NY (US)

(21) Appl. No.: 15/057,358

(22) Filed: Mar. 1, 2016

Related U.S. Application Data
(63) Continuation of application No. 12/988,209, filed on Oct. 15, 2010.
(60) Provisional application No. 61/064,505, filed on Mar. 8, 2008.

(51) Int. Cl. C07D 489/12; A61K 31/485

(52) U.S. Cl. CPC C07D 489/12; A61K 31/485

(57) ABSTRACT

The present invention is directed to oral pharmaceutical compositions of buprenorphine and it pharmaceutically acceptable salts and the use thereof.

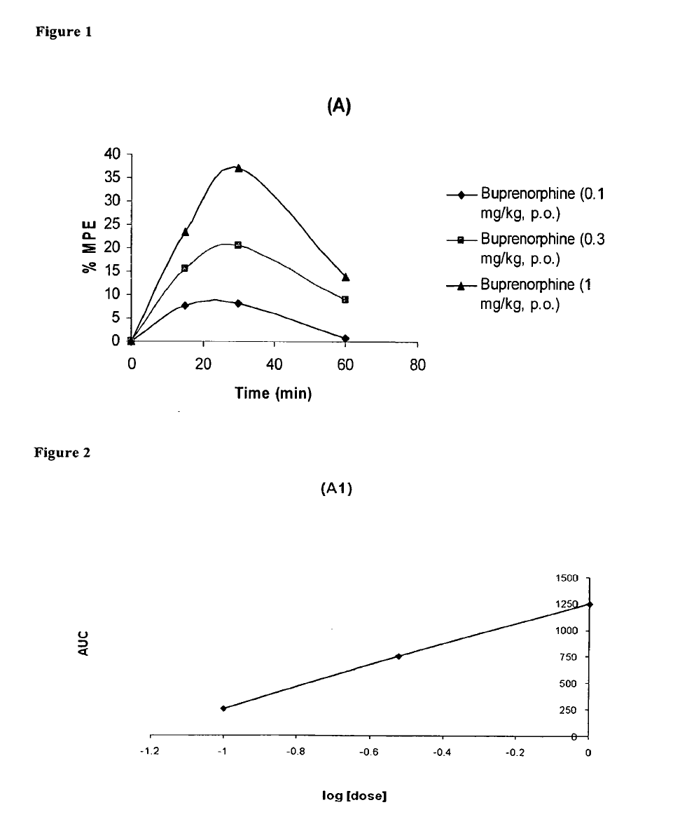

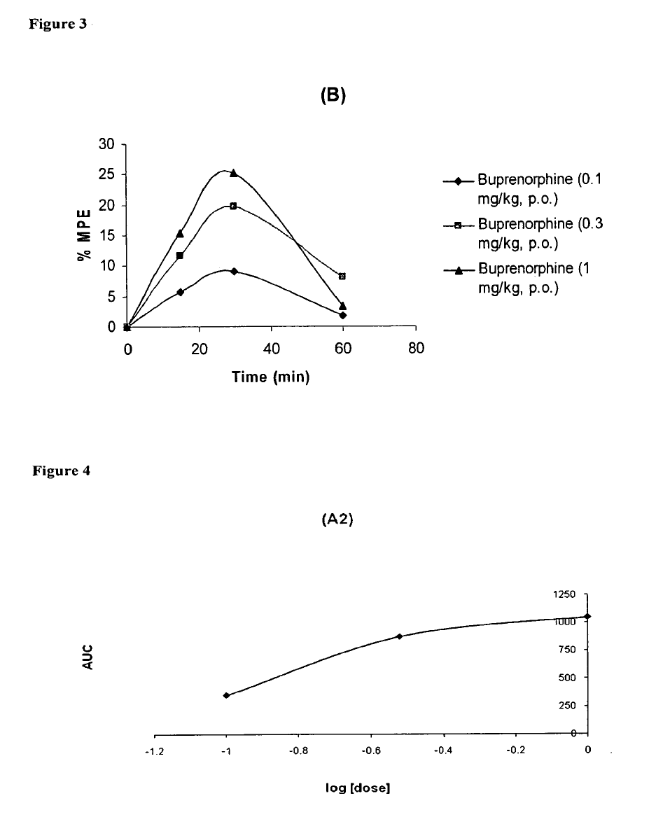

### CROSS-REFERENCES TO RELATED APPLICATIONS

This application is a continuation of co-pending U.S. application Ser. No. 12/988,209 filed Oct. 15, 2010, and a national stage application corresponding to international application PCT/US2009/001502, filed Mar. 9, 2009, which international application is entitled to priority to U.S. provisional patent application No. 61/064,505 filed Mar. 8, 2008.

### BACKGROUND OF THE DISCLOSURE

The present invention is directed to oral pharmaceutical compositions of buprenorphine and it pharmaceutically acceptable salts and the use thereof.

Currently, medical practitioners may choose from several well-accepted classes of pharmaceutical agents in their attempts to alleviate and prevent pain. Examples of agents used include nonsteroidal anti-inflammatory agents (NSAIDs), opioids, cyclooxygenase-2 (COX-2) selective NSAIDs, acetaminophen, tricyclic and non-tricyclic antidepressants, voltage sensitive N-type calcium channel blockers, and alpha adrenergic agonists.

An important goal of analgesic therapy is to achieve continuous relief of pain. Regular administration of an analgesic is generally required to ensure that the next dose is given before the effects of the previous dose have worn off.

Conventional called “immediate-release”, “rapid release” or “short acting”) opioid analgesics have been demonstrated to provide short-lived plasma levels, thereby requiring dosing every 4-6 hours in chronic pain. In contrast, extended release oral opioids are designed to maintain effective plasma levels throughout a 12 or 24-hour dosing interval. Use of extended release opioids can result in fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with short acting opioids.

Clinicians treating cancer pain with opioids have reported significant variability among patients in efficacy and side effects with available opioid analgesics. Patients with poor analgesic efficacy or safety outcomes on one opioid frequently tolerate another opioid well. This clinical observation led to the development of oxycodone ER (OxyContin™). Due to the limitations associated with extended release morphine noted above and the “stigma” associated with its use (i.e., association with addiction, advanced cancer, dying and death), extended release oxycodone gained rapid acceptance by patients with chronic non-cancer pain. However, its widespread use for the treatment of chronic non-malignant pain was also associated with its diversion into the non-medical supply for use both by addicts and recreational drug users

Among the many side effects of opioids are nausea, vomiting, constipation, sedation, fatigue, pruritus, blurred vision, urinary retention, respiratory depression, convulsions, mood changes and alterations of the endocrine and autonomic nervous systems. Many of these side effects are sufficiently bothersome as to require: i) use of additional medications to treat the iatrogenic symptoms; ii) more intensive patient management; iii) use of lower doses that leave patients in continued pain; or iv) in other cases, complete discontinuation of analgesic therapy. Opioids can also produce potentially fatal respiratory depression at high doses.

A number of oral immediate release oral formulations of opioid analgesics have been described in the art, including codeine, hydrocodone, hydromorphone, isomethadone, levorphanol, meperidine, methadone, morphine, oxycodone, oxymorphone, pentazocine, propoxyphene, tapentadol, tramadol, or their pharmaceutically acceptable salts.

A number of oral extended release formulations of opioid analgesics have been developed or commercialized, including morphine, hydromorphone, oxycodone, hydrocodone and oxymorphone.

Buprenorphine or [5α,7α(S)]-17-(Cyclopropylmethyl)-α-(1,1-dimethylethyl)-4,5-epoxy-18,19-dihydo-3-hydroxy-6-methoxy-α-methyl-6,14-ethenomorphinan-7-methanol or its pharmaceutically acceptable salts have been used for the treatment of a variety of medical conditions, including pain and opioid addiction disorders. It is a semi-synthetic opioid first derived in 1966 from thebaine, an alkaloid from the poppy Papaver somniferum.

Buprenorphine is a partial agonist at the mu (μ)-opioid receptor. To our knowledge, no oral extended release formulations of buprenorphine or other partial mu (μ)-opioid opioid analgesics have been developed or commercialized.

Buprenorphine has been commercially available as a parenteral formulation for intravenous and intramuscular use. Buprenorphine has been widely reported to have very poor oral bioavailability and is believed to be ineffective when given orally. For this reason, pharmaceutical companies have made elaborate efforts to develop alternative non-invasive methods of delivering buprenorphine into systemic circulation. Foremost among these methods is the sublingual delivery of buprenorphine for the treatment of pain. This approach has provided only modest efficacy and has been a commercially failure in a number of countries.

Two sublingual formulations of buprenorphine were approved by the FDA in the USA for the “treatment of opioid dependence” but not for pain. One formulation contains buprenorphine alone (Subutex™) and the other contains buprenorphine in combination with naloxone (Suboxone™).

Major disadvantages with sublingual administration of buprenorphine include but are not limited to: (i) highly variable pharmacokinetics and pharmacodynamics; (ii) variability of patient's ability to adhere to the instructions about oral retention of drug; (iii) the development of a depot of buprenorphine on in the oral tissue; (iv) an unpleasant taste and after-taste; (v) a sensation of “gagging”; (vi) durability of a robust effect over the course of 24 hours; and (vii) increased risk of drug abuse through tampering of the dosage form and subsequent intravenous, intranasal and inhalational use.

It is widely recognized that the dose of opioids required to treat pain is highly variable. For example, some patients can be managed with a few mg of morphine while other patients require grams of morphine per day. For this reason, patients receiving opioids are titrated to clinical effect by increasing the dose. An increase in dose should provide a dose proportional increase in bioavailability. Unfortunately, sublingual buprenorphine fails to provide dose proportional bioavailability at particularly high doses, thereby limiting its clinical utility. For example, a 16 mg sublingual dose of buprenorphine provides only 70% dose normalized bioavailability of a 4 mg sublingual dose.

Another limitation with the sublingual route is the high peak concentration of buprenorphine. Both peak concentrations of opioids and the rate of rise in concentrations have variously been implicated in the causation of various opioid side effects such as nausea, drowsiness, dizziness and (acute) cognitive impairment. Orally administered buprenorphine can be formulated to provide significantly lower peak concentrations than sublingual buprenorphine. Such an effect is also desirable in opioid dependent individuals to minimize the “rush” or euphoric effect of high peak concentrations.

There is a need for optimized alternative opioid formulations for the treatment of acute and chronic pain, such as cancer pain and neuropathic pain, and for the treatment of opioid and buprenorphine-responsive conditions other than pain.

There is a need for optimized formulations of non-transdermal opioids that provide reduced pharmacokinetic and pharmacodynamic variability, a rapid onset of effect, a sustained duration of effect, consistent effects over time, improved safety and tolerability. There is also a need for optimized pharmaceutical formulations of oral opioids that deliver the drug into systemic circulation more efficiently and with improved patient and prescriber acceptance.

There is a need for oral (i.e., orally ingested, as opposed to lingual, sublingual or buccal) formulations of buprenorphine that are therapeutically effective for the treatment of various medical conditions, including but not limited to pain and opioid addiction disorders.

There is a need for oral formulations of buprenorphine that are therapeutically efficient and that can provide immediate release and/or sustained release of the buprenorphine.

### DETAILED DESCRIPTION

The applicant has demonstrated that oral administration of buprenorphine can produce robust, dose dependent analgesia.

A variety of opioid analgesics have demonstrated their efficacy in acute pain, chronic cancer and non-cancer pain and in pain states.

The oral route of administration (i.e., oral ingestion) is the most widely used and most widely preferred method of drug administration. It is simple, reliable and readily accessible. Under most conditions of use, particularly outside the hospital setting, it is the recommended method of drug administration. Even in settings of skilled nursing care, where there are technical and human resources to initiate and manage parenteral therapy, the goal is to rapidly transition patients from parenteral medications to oral medications. Some generally cited exceptions to the use of the oral route include: (i) drugs with poor oral bioavailability; (ii) drugs requiring a rapid onset of effect; (iii) where venous access already exists (e.g., in the perioperative or intensive care setting); (iii) where the oral route provides unreliable or inconsistent clinical effects. Oral administration of buprenorphine in immediate release form and in sustained release form has either not been practiced or has been dismissed as unreliable or clinically unacceptable for some of the reasons noted above.

Contrary to this view, the applicant asserts that orally administered buprenorphine can provide acceptable pharmacokinetics, pharmacodynamics, clinical efficacy and safety. Administration of buprenorphine by the oral route provides significantly greater flexibility in dosage form design, clinical utility and patient acceptability. In the USA, buprenorphine is classified as a Schedule III drug. According to the DEA and WHO, Schedule drugs have lower potential for abuse than the drugs in Schedules I and (e.g., fentanyl, codeine, hydrocodone, hydromorphone, methadone, meperidine, morphine, oxycodone, and oxymorphone). In addition, since sustained release drugs are the standard of care for the management of many chronic conditions and sustained release opioids are the standard of care for the management of chronic pain, an orally effective buprenorphine, with its reduced abuse potential compared with Schedule II opioids, has the potential to provide fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with sublingual buprenorphine. Similarly, for the treatment of opioid addiction disorders (where sublingual buprenorphine is approved), oral sustained release buprenorphine can provide similar attributes as seen in patients with pain and has the potential to become the standard of care. When compared with sublingual administration, oral immediate and sustained release buprenorphine may be associated with reduced peak to trough fluctuation in concentrations and clinical effects, such as drug craving. Furthermore, in many cases, such dosage forms have a reduced potential for abuse and diversion than sublingual formulations of buprenorphine which are designed to rapidly dissolve in the oral cavity, thereby reducing subsequent intravenous, intranasal and inhalational abuse.

The present invention is directed at oral pharmaceutical compositions of buprenorphine and their use for the treatment of buprenorphine responsive medical conditions.

The present invention is directed at oral pharmaceutical composition for the treatment of pain, opioid dependence, addiction disorders and other buprenorphine and opioid responsive medical conditions comprising a therapeutically effective amount of buprenorphine or a pharmaceutically acceptable salt of buprenorphine, or a mixture thereof, said dosage form not intended to provide significant oro-mucosal, lingual, sublingual or buccal absorption, said dosage form not intended for oro-mucosal, lingual, sublingual or buccal administration.

The present invention relates to oral buprenorphine pharmaceutical compositions and methods for the treatment of pain, addiction disorders, and other conditions amenable to treatment with buprenorphine or opioid analgesics.

It is an object of certain preferred embodiments of the present invention to substantially improve the efficiency and quality of pain management in human patients experiencing mild to moderate or severe pain.

It is an object of certain preferred embodiments of the present invention to treat pain in patients who have a suboptimal efficacy or safety response with other orally approved opioids, e,g., morphine, codeine, oxycodone, oxymorphone, hydromorphone, methadone, hydrocodone.

It is an object of certain preferred embodiments of the present invention to treat pain in patients who have a suboptimal efficacy or safety response with other orally approved extended release opioids (e.g., MS Contin™, Kadian™, Avinza™, Ultram™ ER, Opana™ ER, Palladone™, Jurnista™).

Specifically excluded from this invention are buprenorphine dosage forms which are administered by the lingual, sublingual, oro-mucosal, transmucosal and buccal routes. Lingual, sublingual, oro-mucosal, transmucosal and buccal routes are intended to provide absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach). Such formulations and their method of administration are well known in the art and include lozenges, transmucosal films, buccal products, mucoretentive products, orally disintegrating tablets, fast dissolving tablets, fast dispersing tablets, fast disintegrating dosage forms, provided they are administered for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain which is unresponsive to other oral formulations of pure or full mu-opioid receptor agonists.

In some preferred embodiments, the in vivo pharmacokinetic parameters of the specifications, embodiments and claims are achieved with oral pharmaceutical compositions comprising a controlled release material to render said dosage form suitable for extended release.

In some preferred embodiments, the specifications and claims are achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine; and a controlled release material with gastroretentive properties to render said dosage form suitable for extended release oral administration to a human patient.

In some preferred embodiments, the dosage form provides an oral pharmaceutical composition comprising a therapeutically effective amount of buprenorphine; and a controlled release material with osmotic release to render said dosage form suitable for extended release oral administration to a human patient.

In some preferred embodiments, the dosage form provides an oral pharmaceutical composition comprising a therapeutically effective amount of buprenorphine; and a controlled release material with zero-order or pseudo-zero-order release to render said dosage form suitable for extended release oral administration to a human patient.

In some preferred embodiments, the oral dosage form of the invention provides an in-vitro release of from about 2% to about 50% by weight of the buprenorphine or a pharmaceutically acceptable salt of buprenorphine from the dosage form at one hour when measured by the USP Basket Method at 100 rpm in 700 ml of Simulated Gastric Fluid (SGF) at 37° C. In other preferred embodiments, under the same dissolution conditions, said dosage form provides an in-vitro release rate by weight of the buprenorphine or a pharmaceutically acceptable salt of buprenorphine from the dosage form at one hour from about 2% to about 45%, or from about 2% to about 60%, or from about 5% to about 40%, or from about 5% to about 60%, or from about 10% to about 70%, or from about 10% to about 80%, or from about 15% to about 90%, or from about 60 to about 100%, or from about 80 to about 100%, or greater than about 1%, or greater than about 5%, or greater than about 15%, or greater than about 40%, or greater than about 60%, or greater than about 80%, or greater than about 90%, or greater than about 95%.

The amount of buprenorphine in the oral dosage form will vary depending on variety of physiologic, pharmacologic, pharmacokinetic, pharmaceutical and physicochemical factors, including: (i) the choice of buprenorphine as the base, pharmaceutically acceptable salt or mixtures therof; (ii) the nature of the oral dosage form (e.g, immediate release or extended release); (iii) the anatomical location of the pain relieving target; (iv) the intensity and intractability of the pain; (v) the contribution of different mechanism to the initiation, propagation, summation and maintenance of the pain; (vi) the absorption, metabolism, distribution and excretion of orally administered buprenomhine in healthy subjects and in patients with various diseases and disorders, including renal and hepatic impairment; (vii) the presence of comorbid pathology; (viii) the patient's risk of iatrogenic side effects; (ix) the tolerability of the dose, including the patient's propensity for buprenorphine associated CNS and gastrointestinal side effects; (x) use of concurrent analgesics; (xi) the efficiency of the dosage form.

The invention is also directed to methods of preparing the dosage forms disclosed herein.

In certain preferred embodiments, the buprenorphine in the dosage form is combined with one or more other drugs for the treatment of the same medical condition as the buprenorphine or for the treatment of a different medical condition. All modes of co-administration are contemplated, including via an oral, subcutaneous, direct intravenous, slow intravenous infusion, continuous intravenous infusion, intravenous or epidural patient controlled analgesia (PCA and PCEA), intramuscular, intrathecal, epidural, intracisternal, intramuscular, intraperitoneal, transdermal, topical, transmucosal, buccal, sublingual, inhalation, intranasal, epidural, intra-atricular, intranasal, rectal or ocular routes.

In certain preferred embodiments of the present invention, an effective amount of buprenorphine in immediate release form is included in the controlled release unit dose buprenorphine formulation to be administered. The immediate release form of the buprenorphine is preferably included in an amount which is effective to shorten the time to Cₘₐₓ of the buprenorphine in the blood (e.g., plasma). In such embodiments, an effective amount of the buprenorphine in immediate release form may be coated onto the substrates of the present invention. For example, where the extended release buprenorphine from the formulation is due to a controlled release coating, the immediate release layer would be overcoated on top of the controlled release coating. On the other hand, the immediate release layer maybe coated onto the surface of substrates wherein the buprenorphine is incorporated in a controlled release matrix. Where a plurality of the sustained release substrates comprising an effective unit dose of the buprenorphine (e.g., multiparticulate systems including pellets, spheres, beads and the like) are incorporated into a hard gelatin capsule, the immediate release portion of the buprenorphine dose may be incorporated into the gelatin capsule via inclusion of the sufficient amount of immediate release buprenorphine as a powder or granulate within the capsule. Alternatively, the gelatin capsule itself may be coated with an immediate release layer of the buprenorphine. One skilled in the art would recognize still other alternative manners of incorporating the immediate release buprenorphine into the unit dose. Such alternatives are deemed to be encompassed by the appended claims. By including such an effective amount of immediate release buprenorphine in the unit dose, the experience of relatively higher levels of pain in patients may be significantly reduced.

In certain preferred embodiments, the amount of buprenorphine in the dosage form is about 0.001 mg to 1500 mg. In other more preferred embodiments, the amount of buprenorphine in the dosage form is about 0.1 mg to 1000 mg. In most preferred embodiments, the amount of buprenorphine in the dosage form is about 0.5 mg to about 500 mg or about 1 mg to about 200 mg, or 2 mg to about 100 mg or 1 mg to about 60 mg.

The term “USP Paddle or Basket Method” is the Paddle and Basket Method described, e.g., in specified in the United States Pharmacopeia, USP-28 NF-23 (2005), published by the United States Pharmacopeial Convention, Inc, herein incorporated by reference.

The term “bioavailability” is defined for purposes of the present invention as the extent to which the drug (e.g., buprenorphine) is absorbed from the unit dosage forms.

As used herein with respect to the buprenorphine dosage form of the invention, the term “oral”, “oral dosage form”, “oral pharmaceutical dosage form”, “oral administration”, and “oral route” and the like all refer to any method of administration through the mouth for rapid deposit into the stomach or alimentary canal. The oral dosage form of the invention is usually ingested intact, although it may be ingested un-intact or tampered (e.g., crushed) and usually with the aid of water or a beverage to hasten passage through the mouth. Specifically excluded from this invention and the forgoing definition of oral dosage forms are buprenorphine dosage forms and pharmaceutical compositions which are administered by the lingual, sublingual, oro-mucosal, transmucosal and buccal routes. Lingual, sublingual, oro-mucosal, transmucosal and buccal routes are intended to provide absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach). Such formulations and their method of administration are well known in the art and include lozenges, transmucosal films, buccal products, mucoretentive products, orally disintegrating tablets, fast dissolving tablets, fast dispersing tablets, fast disintegrating dosage forms, provided they are administered for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

All oral pharmaceutical dosage forms of the invention are contemplated, including oral suspensions, tablets, capsules, effervescent tablets, effervescent powders, powders, solutions, powders for reconstitution, oral gastroretentive tablets and capsules, administered as immediate release, modified release, enteric coated, sustained release, controlled release, pulsatile release and extended release dosage form.

As used herein, “controlled release” is interchangeable with “extended release”, “sustained release”, “modified release”, “delayed release” and the like.

Controlled release dosage forms of the present invention release of buprenorphine from the oral dosage form at slower rate than immediate release formulations. In some preferred embodiments, controlled release dosage forms of release buprenorphine at such a rate that blood (e.g., plasma) concentrations (levels) or therapeutic effects are maintained within the therapeutic range (above the minimum effective therapeutic concentration) but below toxic levels for intended duration e.g., over a period of 1 to 24 hours, preferably over a period of time indicative of a Q3, Q4, Q6, Q8, Q12 or Q24H administration). Notwithstanding the foregoing, in some preferred embodiments, the controlled release formulations of the present invention provide therapeutic effects for a duration that is longer or substantially longer than the duration of meaningful or detectable plasma concentrations of buprenorphine.

The term “immediate release buprenorphine” for purposes of the present invention, is buprenorphine for oral administration in a dosage form which is formulated to release the active drug from the dosage form immediately (i.e., without an attempt to delay or prolong the release of the active drug from the dosage form as is the case for extended release dosage forms). In the absence of a commercially available oral immediate release buprenorphine product, an available parenteral formulation of buprenorphine or a salt thereof may be used orally or a solution of buprenorphine or a salt thereof may be prepared for the purpose of in vivo testing requiring immediate release buprenorphine.

For purposes of the invention, the oral controlled release formulations disclosed herein and the oral immediate release control formulations are dose proportional. In such formulations, the pharmacokinetic parameters (e.g., AUC and Cₘₐₓ) generally increase linearly from one dosage strength to another. Therefore the pharmacokinetic parameters of a particular dose can be inferred from the parameters of a different dose of the same formulation.

The term “agonist” means a ligand that binds to a receptor and alters the receptor state resulting in a biological response. Conventional agonists increase receptor activity, whereas inverse agonists reduce it.

The term “opioid agonist” means a molecule that causes a specific physiologic, pathophysiologic or pharmacologic effect after binding to an opioid receptor.

An “antagonist” is a drug or ligand that reduces the action of another drug or ligand, generally an agonist. Many antagonists act at the same receptor macromolecule as the agonist.

The term “receptor” means a molecule within a cell, on a cell surface, on a membrane, in tissue, in fluid or otherwise found in humans that serve as a recognition or binding site to cause specific physiologic, pathophysiologic or pharmacologic effects. The term “receptor” also means a cellular macromolecule, or an assembly of macromolecules, that is concerned directly and specifically in chemical signaling between and within cells. Combination of a hormone, neurotransmitter, drug, ligand, or intracellular messenger with its receptor(s) initiates a change in cell function.

The term “opioid receptor” includes mu (μ), delta (δ) and kappa (κ) opioid receptors, their subtypes and splice variants such as mu₁, mu₂, delta₁, delta₂, kappa₁, kappa₂ and kappa₃, etc.

Opioid antagonists are known or readily determined by individuals who practice the art. Preferably, the opioid antagonists useful for the present invention may be selected from the group consisting of naltrexone, methylnaltrexone, nalbuphine, naloxone, nalmefene, cyclazocine, cyclorphan, oxilorphan nalorphine, nalorphine dinicotinate, nalmefene, nadide and levallorphan.

In certain preferred embodiments of the present invention, the invention allows for the use of lower doses of buprenorphine by virtue of the inclusion or co-administration of an additional drug for the prevention or treatment of pain. By using lower amounts of either or both drugs, the side effects associated with treatment in humans are reduced.

The term “buprenorphine” means buprenorphine base, as well as their pharmaceutically acceptable salts, prodrugs, esters, analogs, derivatives, solvates, complexes, polymorphs, and hydrates, as racemates or an individual diastereoisomers or enantiomeric isomers thereof or mixtures thereof. In some preferred embodiments, the dosage form comprises buprenorphine base or their pharmaceutically acceptable salts, or mixtures thereof. In some even more preferred embodiments, the dosage form comprises buprenorphine base or buprenorphine HCl, or mixtures thereof.

The phrase “comprising a therapeutically effective amount of buprenorphine” means “comprising a therapeutically effective amount of buprenorphine or a pharmaceutically acceptable salt of buprenorphine, or prodrugs, esters, analogs, derivatives, solvates, complexes, polymorphs and hydrates thereof, as racemates or an individual diastereoisomers or enantiomeric isomers thereof or mixtures thereof.

When the dosage form includes a pharmaceutically acceptable salt, any salt may be use. Preferably, the salt is the hydrochloride salt of buprenorphine.

The singular forms “a,” “an” and “the” include plural referents unless the context clearly dictates otherwise. Thus, for example, reference to “a polymer” includes a single polymer as well as a mixture of two or more different polymers, reference to “a permeation enhancer” includes a single permeation enhancer as well as two or more different permeation enhancer in combination, and the like.

In some embodiments, the dosage form of the invention, one or more or all of the specifications and claims applicable to the prevention and treatment of pain or addiction disorders is also applicable to the prevention or treatment of any other disease or disorder that responds to opioid agonists or to buprenorphine.

The prevention and treatment of all diseases and disorders is contemplated by the use of this invention, including without limitation, (i) pain; (ii) addiction disorders; (iii) opioid substitution and opioid maintenance therapy; (iv) restless leg syndrome; (v) cough; (vi) urinary incontinence; (vii) fibromyalgia; (viii) pain associated with sickle cell disease, including vaso-occlusive crisis; (ix) peripheral and central neuropathic pain; (x) cancer pain; (xi) breakthrough pain; (xii) visceral pain; (xiii) dyspnea and respiratory distress; (xiv) infectious, immunologic, cardiovascular, pulmonary, gastrointestinal, hepatic, biliary, nutritional, metabolic, endocrine, hematologic, oncologic, musculoskeletal, rheumatic, neurologic, psychiatric, genitourinary, gynecologic, obstetric, pediatric, otolaryngogologic, ophthalmic, dermatologic, dental, oral, and genetic disorders, diseases and maladies and signs and symptoms thereof; (xv) depression, schizophrenia, influenza, common colds, anxiety, panic attacks, agoraphobia, ADHD, insomnia, sleep disorders, nasal congestion, headaches, migraine, urinary incontinence, constipation, allergies, cough, pneumonia, COPD, asthma, fluid retention, acid reflux, peptic ulcers, hypertension, cardiac arrhythmias, hypercholesterolemia, CHF, fever, diarrhea, back pain, myofascial pain, osteoarthritis, neuropathic pain, cancer pain, acute pain, diabetes, muscle spasms, and rheumatoid arthritis, and signs and symptoms thereof; and (xvi) disorders, diseases and maladies, and signs and symptoms thereof referred to in Harrison's Principles of internal Medicine, 16th Edition, 2004, Kasper D L, Braunwald W, Fauci A, Hauser S, Longo D, and Jameson J L (eds)], which is hereby incorporated in its entirety by reference; said disorders, diseases and maladies, and signs and symptoms thereof comprising buprenorphine responsive medical conditions.

In some preferred embodiments, the oral pharmaceutical dosage forms of buprenorphine are used to treat pain, cough, dyspnea, opioid addiction disorders, restless leg syndrome, fibromyalgia, acute herpes zoster, visceral pain, breakthrough pain, opioid dependence and urinary incontinence.

As used herein, the term “pain” includes: (i) peripheral neuropathic pain, e.g., acute and chronic inflammatory demeyelinating polyradiculopathy, alcoholic polyneuropa thy, chemotherapy-induced polyneuropathy, complex regional pain syndrome (CRPS) Type I and Type II, entrapment neuropathies (e.g., carpal tunnel syndrome), HIV sensory neuropathy, iatrogenic neuralgias (e.g., postthoracotomy pain, postmastectomy pain), idiopathic sensory neuropathy, painful diabetic neuropathy, phantom limb pain, postherpetic neuralgia, trigeminal neuralgia, radiculopathy (e.g., cervical thoracic, lumbosacral), sciatica, acute herpes zoster pain, temporomandibular joint disorder pain and postradiation plexopathy; and (ii) central neuropathic pain, e.g., compressive myelopathy from spinal stenosis, HIV myelopathy, multiple sclerosis pain, Parkinson's disease pain, postischemic myelopathy, post postradiation myelopathy, poststroke pain, posttraumatic spinal cord injury and syringomyelia; and (iii) cancer associated neuropathic pain, e.g., chemotherapy induced polyneuropathy, neuropathy secondary to tumor infiltration or nerve compression, phantom breast pain, postmastectomy pain, postradiation plexopathy and myelopathy; (iv) chronic pain, e.g., back pain, rheumatoid arthritis, osteoarthritis, inflammatory pain, non-inflammatory pain, myofascial pain, fibromyalgia, cancer pain, visceral pain, somatic pain, pelvic pain, musculoskeletal pain, post-traumatic pain, bone pain and idiopathic pain; (v) acute pain, e.g, acute postsurgical pain (including laparoscopic, laparatomy, gynecologic, urologic, cardiothoracic, arthroscopic, gastrointestinal, neurologic, orthopedic, oncologic, maxillofacial, ophthalmic, otolaryngologic, soft tissue, plastic, cosmetic, vascular and podiatric surgery, including abdominal surgery, abdominoplasty, adenoidectomy, amputation, angioplasty, appendectomy, arthrodesis, arthroplasty, arthroscopy, bilateral cingulotomy, biopsy, brain surgery, breast biopsy, cauterization, cesarean section, cholecystectomy, circumcision, commissurotomy, cordotomy, corneal transplantation, cricothoracotomy, discectomy, diverticulectomy, episiotomy, endarterectomy, endoscopic thoracic sympathectomy, foreskin restoration, fistulotomy, frenectomy, frontalis lift, flindectomy, gastrectomy, grafting, heart transplantation, hemicorporectomy, hemorrhoidectomy, hepatectomy, hernia repair, hypnosurgery, hysterectomy, kidney transplantation, laminectomy, laparoscopy, laparotomy, laryngectomy, lithotripsy, lobotomy, lumpectomy, lung transplantation, mammectomy, mammoplasty, mastectomy, mastoidectomy, mentoplasty, myotomy, mryingotomy, nephrectomy, nissen fundoplication, oophorectomy, orchidectomy, parathyroidectomy, penectomy, phalloplasty, pneumotomy, pneumonectomy, prostatectomy, psychosurgery, radiosurgery, ritidoplasty, rotationplasty, sigmoidostomy, sphincterotomy, splenetomy, stapedectomy, thoracotomy, thrombectomy, thymectomy, thyroidectomy, tonsillectomy, tracheotomy, tracheostomy, tubal ligation, ulnar collateral ligament reconstruction, ureterosigmoidostomy, vaginectomy, vasectomy, vulvectomy; renal colic; incisional pain; inflammatory incisional pain; nociceptive incisional pain; acute neuropathic incisional pain following surgery), renal colic, trauma, acute back pain, burn pain, burn dressing change pain, migraine pain, tension headache pain, acute musculoskeletal pain, acute exacerbation or flare of chronic back pain, acute exacerbation or flare of osteoarthritis, acute exacerbation or flare of chronic pain, breakthrough chronic non-cancer pain, breakthrough cancer pain, acute exacerbation or flare of fibromylagia, acute exacerbation or flare of rheumatoid arthritis, acute exacerbation or flare of myofascial pain, acute exacerbation or flare of chronic idiopathic pain, acute exacerbation or flare of neuropathic pain, procedure related pain (e.g., arthroscopy, laparoscopy, endoscopy, intubation, bone marrow biopsy, soft tissue biopsy, catheterization), and other self-limiting pain states.

As used herein, the term “acute pain” refers to self-limiting pain that subsides over time and usually lasting less that about 30 days and more preferably tasting less than about 21 days. Acute pain does not include chronic conditions such as chronic neuropathy, chronic neuropathic pain and chronic cancer and non-cancer pain.

As used herein, “neuropathic pain” is pain initiated or caused by a primary lesion or dysfunction of the nervous system and includes (i) peripheral neuropathic pain and (ii) central neuropathic pain.

As used herein, the term “chronic pain” includes all non-neuropathic pain usually lasting more than 30 days, including inflammatory pain, non-inflammatory pain, muscle pain, joint pain, fascia pain, visceral pain, bone pain and idiopathic pain.

The term “analgesic effectiveness” is defined for purposes of the present invention as a satisfactory prevention, reduction in or elimination of pain, along with a tolerable level of side effects, as determined by the human patient.

According to the American Academy of Pain Medicine, the American Pain Society and the American Society of Addiction Medicine “addiction” and “addiction disorder” is a primary, chronic, neurobiologic disease, with genetic, psychosocial, and environmental factors influencing its development and manifestations. It is characterized by behaviors that include one or more of the following: impaired control over medication use, compulsive use, continued use despite harm, and craving. The pharmaceutical composition of the present invention is in some embodiments intended to treat addiction disorder, particularly opioid addiction disorder and poly-substance abuse involving opioids. In some embodiments, the dosage form of the invention is intended to reduce or eliminate the craving or desire for opioids and the antisocial, medically harmful and potentially criminal behavior of the patient with the addiction disorder. The use of sublingual buprenorphine for the treatment of addiction disorder has been well established in the literature.

The term “therapeutic effectiveness” is defined for purposes of the present invention as a satisfactory prevention, reduction in or elimination of signs and symptoms of the medical disorder, disease or syndrome (e.g., pain, addiction disorder), along with a tolerable level of side effects, as determined by the human patient.

“Pharmaceutically or therapeutically acceptable excipient or carrier” or “excipient” refers to a substance which does not interfere with the effectiveness or the biological activity of the active ingredients and which is not toxic to the subject. In some embodiments of the present invention, pharmaceutically or therapeutically acceptable excipients or carriers may play a role in imparting or optimizing the rate and extent of absorption or buprenorphine or additional drugs in the pharmaceutical composition. In some embodiments of the present invention, pharmaceutically or therapeutically acceptable excipients or carriers may play a role in stabilizing the buprenorphine or additional drugs in the pharmaceutical composition.

In one embodiment of the invention, the dosage form includes both an immediate release and extended release component.

In one embodiment of the invention, the dosage form includes a capsule within a capsule, each capsule containing a different drug or the same drug intended for treating the same or a different malady. In some preferred embodiments, the outer capsule may be an enteric coated capsule or a capsule containing an immediate release formulation to provide rapid plasma concentrations or a rapid onset of effect or a loading dose and the inner capsule contains an extended release formulation. In some preferred embodiments, up to 3 capsules within a capsule are contemplated as part of the invention. In one embodiment, of the invention, the dosage form involves one or more tablets within a capsule, wherein the buprenorphine is either in the tablet and/or in one of the capsules.

In one embodiment of the invention, the formulation is ingested orally as a tab capsule, preferably as a capsule.

“Therapeutically effective amount” or “therapeutically-effective” refers to the amount of an active agent sufficient to induce a desired biological result. That result may be alleviation of the signs, symptoms, or causes of a disease, or any other desired alteration of a biological system.

“Therapeutically effective amount of buprenorphine” refers to the amount of oral buprenorphine sufficient to prevent, to cure, or at least partially arrest a medical disorder, disease, sign or symptom for which the buprenorphine has been prescribed to a subject.

The term “effective amount” means the quantity of a compound according to the invention necessary to prevent, to cure, or at least partially arrest a medical disorder, disease, sign or symptom for which the buprenorphine has been prescribed to a subject.

The term “pharmaceutically acceptable salt” as used herein refers to a salt which is toxicologically safe for human and animal administration. Nonlimiting examples of salts include hydrochlorides, hydrobromides, hydroiodides, sulfates, bisulfates, nitrates, citrates, tartrates, bitartrates, phosphates, malates, maleates, napsylates, fumarates, succinates, acetates, terephlhalates, pamoates and pectinates.

It is contemplated that the present invention may be used alone or in combination with other drugs to provide additive, complementary, or synergistic therapeutic effects or for the treatment of entirely different medical conditions.

In some embodiments, the oral buprenorphine is intended to prevent or treat pain. A co-administered drug (in the same or different dosage form, by any route of administration) may be used to provide additive, complementary, superadditive or synergistic therapeutic analgesic effects, including other NSAIDs, NO-NSAIDs, COX-2 selective inhibitors, acetaminophen, tramadol, local anesthetics, antidepressants, beta adrenergic agonists, alpha-2 agonists, selective prostanoid receptor antagonists, cannabinoid agonists, other opioid receptor agonists, NMDA receptor antagonists, gabapentin, pregabalin, gabapentinoids, neuronal nicotinic receptor agonists, calcium channel antagonists, sodium channel blockers, superoxide dismutase mimetics, p38 MAP kinase inhibitors, TRPV1 agonists, dextromethorphan, dextrorphan, ketamine, glycine receptor antagonists, antiepileptics, and any other drugs that can be shown by a person proficient in the art to prevent or treat pain.

Particularly preferred combinations include buprenorphine with other opioids.

Opioid agonists include alfentanil, allylprodine, alphaprodine, anileridine, apomorphine, apocodeine, benzylmorphine, bezitramide, buprenorphine, butorphanol, carfentanil, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethytmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, nociceptin/orphanin FQ (N/OFQ), normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, racemorphan, remifentanil, sufentanil, tapentadol, tramadol, tilidine, methylnaltrexone, naloxone methiodide, naloxonazine, nalmexone, nalbuphine, nalorphine dinicotinate, naltrindole (NTI), naltrindole isothiocyanate, (NTH), naltriben (NTB), nor-binaltorphimine (nor-BNI), beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69,593, spiradoline, DPDPE, [D-Ala2,Glu4] deltorphin, DSLET, Met-enkephalin, Leu-enkephalin, (3-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmaceutically acceptable salts, prodrugs, esters, analogs, derivatives, solvates, complexes, polymorphs, hydrates and metabolites, as racemates or an individual.

Methods of Carrying Out the Invention

Dosage Forms

Pharmaceutical composition and methods of the present invention contain buprenorphine base or pharmaceutically acceptable salts in racemic or enantiomeric form, or mixtures thereof intended for oral administration.

All oral pharmaceutical dosage forms of the invention are contemplated, including oral suspensions, tablets, capsules, lozenges, effervescent tablets, effervescent powders, powders, solutions, powders for reconstitution, gastroretentive tablets and capsules, orally disintegrating tablets, oral fast dissolving tablets, oral fast dispersing tablets, oral fast disintegrating dosage forms, each administered as immediate release, modified release, enteric coated, sustained release, controlled release, pulsatile release or extended release dosage form.

The formulation may optionally comprise excipients. Non-limiting examples of these auxiliary materials (or pharmaceutically acceptable excipients) are (i) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like; (ii) Disintegrants such as starches, pregelatinized corn starch, pregelatinized starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations; (iii) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like; (iv) Stabilizers such as any antioxidation agents, buffers, or acids, and the like.; (v) Lubricants such as magnesium stearate, calcium hydroxide, talc, colloidal silicon dioxide, sodium stearyl fumarate, hydrogenated vegetable oil, stearic acid, glyceryl behenate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like; (vi) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like; (vii) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like; (viii) Anti-adherents or glidants such as talc, corn starch, DL-leucine, sodium lauryl sulfate, and magnesium, calcium, or sodium stearates, and the like; (ix) Pharmaceutically compatible carriers such as acacia, gelatin, colloidal silicon dioxide, calcium glycerophosphate, calcium actate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, or pregetatinized starch, and the like; and (x) exipients referred to herein.

Oral Immediate Release Dosage Forms

Pharmaceutical composition and methods of the present invention contain buprenorphine base or pharmaceutically acceptable salts in racemic or enantiomeric form, or mixtures thereof in and they are intended for oral administration.

All oral immediate release pharmaceutical dosage forms of the invention are contemplated. The preparation of oral immediate release dosage forms has been described in the art. A majority of oral dosage forms commercially available world-wide are formulated as immediate release products.

Controlled-Release Dosage Forms

All oral extended release pharmaceutical dosage forms of the invention are contemplated. The preparation of oral extended release pharmaceutical dosage forms has been described in the art.

The controlled-release dosage form may optionally include a controlled release material which is incorporated into a matrix along with the buprenorphine, or which is applied as a sustained release coating over a substrate comprising the drug (the term “substrate” encompassing beads, pellets, spheroids, tablets, tablet cores, etc). The controlled release material may be hydrophobic or hydrophilic as desired. The oral dosage form according to the invention may be provided as, for example, granules, spheroids, pellets or other multiparticulate formulations. An amount of the multiparticulates which is effective to provide the desired dose of buprenorphine over time may be placed in a capsule or may be incorporated in any other suitable oral solid form, e.g., compressed into a tablet. On the other hand, the oral dosage form according to the present invention may be prepared as a tablet core coated with a controlled-release coating, or as a tablet comprising a matrix of drug and controlled release material, and optionally other pharmaceutically desirable ingredients (e.g., diluents, binders, colorants, lubricants, etc.). The controlled release dosage form of the present invention may also be prepared as a bead formulation or an osmotic dosage formulation.

In certain preferred embodiments of the present invention, the controlled-release formulation is achieved via a matrix (e.g. Ea matrix tablet) which includes a controlled-release material as set forth below. A dosage form including a controlled-release matrix provides in-vitro dissolution rates of buprenorphine within the preferred ranges and that releases the buprenorphine in a pH-dependent or pH-independent manner. The materials suitable for inclusion in a controlled-release matrix will depend on the method used to form the matrix. The oral dosage form may contain between 1% and 99% (by weight) of at least one hydrophilic or hydrophobic controlled release material.

A non-limiting list of suitable controlled-release materials which may be included in a controlled-release matrix according to the invention include hydrophilic and/or hydrophobic materials, such as gums, cellulose ethers, acrylic resins, protein derived materials, waxes, shellac, and oils such as hydrogenated castor oil, hydrogenated vegetable oil hydrogenated Type I or Type II vegetable oils, polyoxyethylene stearates and distearates, glycerol monostearate, and non-polymeric, non-water soluble liquids carbohydrate-based substances or poorly water soluble, high melting point (mp=40 to 100° C.) waxes and mixtures thereof.

Hydrogenated vegetable oils of the present invention may include hydrogenated cottonseed oil (e.g., Akofine®; Lubritab®; Sterotex® NF), hydrogenated palm oil (Dynasan® P60; Softisan® 154), hydrogenated soybean oil (Hydrocote®; Lipovol HS-K®; Sterotex® HM) and hydrogenated palm kernel oil (e,g., Hydrokote® 112).

Polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g, Lipo-PEG® 100-S; Myrj® 59; Hodag® 600-S; Ritox®59; Hodag® 22-S; PEG-4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment of the present invention, the buprenorphine is combined with beeswax, hydroxypropyl methyl cellulose (e.g, HPMC K15M), silicon dioxide (alone or in combination with Al₂O₃; e.g, Aerosil®, Aerose® 200, Aerosil® COK84).

In one embodiment of the present invention, the buprenorphine is combined with hydrogenated cottonseed oil (e.g., Sterotex® NF), hydroxypropyl methyl cellulose (e.g, HPMC K15M), coconut oil and silicon dioxide (alone or in combination with Al₂O₃; e.g, Aerosil®, Aerosil® 200, Aerosil® COK84).

In another embodiment of the present invention, the buprenorphine is combined with glycerol monostearate (e.g., Cithrol® GMS), hydroxypropyl methyl cellulose (e.g, HPMC K100M) and silicon dioxide (alone or in combination with Al₂O₃; e.g, Aerosil®, Aerosil® 200, Aerosil® COK84).

In yet another embodiment of the present invention, the buprenorphine is combined with hydrogenated palm kernel oil (e.g., Hydrokote® 112), hydroxypropyl methyl cellulose (e.g. HPMC K15M) and silicon dioxide (alone or in combination with Al₂O₃; e.g. Aerosil®, Aerosil® 200, Aerosil® COK84).

In one embodiment of the present invention, release rate modifiers, including hydroxypropyl methyl cellulose (e.g, HPMC K15M) may incorporated. Release rate modifiers can also have additional useful properties that optimize the formulation.

A variety of agents may be incorporated into the invention as thixotropes (e.g., fumed silicon dioxides, Aerosil®, Aerosil® COK84, Aerosil® 200, etc.) Thixotropes enhance the pharmaceutical formulations of the invention by increasing the viscosity of solutions complementing the action of HPMCs.

Any pharmaceutically acceptable hydrophobic or hydrophilic controlled-release material which is capable of imparting controlled-release of the buprenorphine may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Certain preferred embodiments utilize mixtures of any of the foregoing controlled-release materials in the matrices of the invention.

The matrix also may include a binder. In such embodiments, the binder preferably contributes to the controlled-release of the buprenorphine from the controlled-release matrix.

Preferred hydrophobic binder materials are water-insoluble with more or less pronounced hydrophilic and/or hydrophobic trends. Preferred hydrophobic binder materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. Preferably, the hydrophobic binder materials useful in the invention have a melting point from about 30 to about 200° C., preferably from about 45 to about 90° C. When the hydrophobic material is a hydrocarbon, the hydrocarbon preferably has a melting point of between 25 and 90° C. Of the long chain (C₈-C₅₀) hydrocarbon materials, fatty (aliphatic) alcohols are preferred. The oral dosage form may contain up to 98% (by weight) of at least one digestible, long chain hydrocarbon.

The oral dosage form contains up to 98% (by weight) of at least one polyalkylene glycol. The hydrophobic binder material may comprise natural or synthetic waxes, fatty alcohols (such as lauryl, myristyl, stearyl, cetyl or preferably cetostearyl alcohol), fatty acids, including hut not limited to fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C.

In certain preferred embodiments, a combination of two or more hydrophobic binder materials are included in the matrix formulations. If an additional hydrophobic binder material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable controlled-release matrix comprises at least one water soluble hydroxyalkyl cellulose, at least one C₁₂-C₃₆, preferably C₁₄-C₂₂, aliphatic alcohol and, optionally, at least one polyalkylene glycol. The hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present oral dosage form will be determined, inter alia, by the precise rate of the buprenorphine release required. The aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of aliphatic alcohol in the present oral dosage form will be determined, as above, by the precise rate of the buprenorphine release required. It will also depend on whether at least one polyalkylene glycol is present in or absent from the oral dosage form. In the absence of at least one polyalkylene glycol, the oral dosage form preferably contains between 20% and 50% (by wt) of the aliphatic alcohol. When a polyalkylene glycol is present in the oral dosage form, then the combined weight of the aliphatic alcohol and the polyalkylene glycol preferably constitutes between 20% and 50% (by wt) of the total dosage.

In one preferred embodiment, the ratio of e.g., the at least one hydroxyalkyl cellulose or acrylic resin to the at least one aliphatic alcohol/polyalkylene glycol determines, to a considerable extent, the release rate of the buprenorphine from the formulation. A ratio of the hydroxyalkyl cellulose to the aliphatic alcohol/polyalkylene glycol of between 1:2 and 1:4 is preferred, with a ratio of between 1:3 and 1:4 being particularly preferred.

The polyalkylene glycol maybe, for example, polypropylene glycol or, which is preferred, polyethylene glycol. The number average molecular weight of the at least one polyalkylene glycol is preferred between 1,000 and 15,000 especially between 1,500 and 12,000.

Another suitable controlled-release matrix comprises an alkylcellulose (especially ethylcellulose), a C₁₂ to C₃₆ aliphatic alcohol and, optionally, a polyalkylene glycol.

Another method of producing the dosage form of the invention involves liquid fill compositions, including hydrogenated Type I or Type II vegetable oils (e.g., Hydrokote™ 112), polyoxyethylene stearates and distearates, glycerol monostearate (e.g., Cithrol™ GMS), non-polymeric, non-water soluble liquids carbohydrate-based substances, poorly water soluble, high melting point (mp=40 to 100° C.) waxes.

Hydrogenated vegetable oils may include hydrogenated cottonseed oil (e.g., Akofine™; Lubritab™; Sterotex™ NF), hydrogenated palm oil (Dynasan™ P60; Softisan™ 154), hydrogenated soybean oil (Hydrocote™; Lipovol HS-K™ ; Sterotex™ HM) and hydrogenated palm kernel oil (e.g., Hydrokote™ 112).

Polyoxyethylene stearates and distearates may include Polyoxyl 2, 4, 6, 8, 12, 20, 30, 40, 50, 100 and 150 stearates e.g., Hodag™ DGS; PEG-2 stearate; Acconon™ 200-MS; Hodag™ 20-S; PEG-4 stearate; Cerasynt™ 616; Kessco™ PEG 300 Monostearate; Acconon™ 400-MS; Cerasynt™ 660; Cithrol™ 4MS; Hodag™ 60-S; Kessco™ PEG 600 Monostearate; Cerasynt™ 840; Hodag 100-S; Myrj™ 51; PEG-30 stearate; polyoxyethylene (30) stearate; Crodet™ S40; E431; Emerest™ 2672; Atlas G-2153; Crodet™ S50) and polyoxyl 4, 8, 12, 32 and 150 distearates (e.g, Lipo-PEG™ 100-S; Myrj™ 59; Hodag™ 600-S; Ritox™ 59; Hodag™ 22-S; PEG-4 distearate; Hodag™ 42-S; Kessco™ PEG 400 DS; Hodag™ 62-S; Kessco™ PEG 600 Distearate; Hodag™ 154-S; Kessco™ PEG 1540 Distearate; Lipo-PEG™ 6000-DS; Protamate™ 6000-DS).

In one embodiment of the present invention, release rate modifiers, including hydroxypropyl methyl cellulose (e.g, HPMC K15M) may be incorporated. Release rate modifiers can also have additional useful properties that optimize the formulation.

A variety of agents may be incorporated into the invention as thixotropes (e.g., fumed silicon dioxides, Aerosil™, Aerosil™ COK84, Aerosil™ 200, etc.). Thixotropes enhance the pharmaceutical formulations of the invention by increasing the viscosity of solutions during attempted extraction, complementing the action of HPMCs. They may also provide a tamper resistance by helping to retain the structure of dosage units that have been heated to temperatures greater than the melting point of the base excipient (Aerosils are unaffected by heat).

In addition to the above ingredients, a controlled-release matrix may also contain suitable quantities of other materials, e.g., diluents, lubricants, binders, granulating aids, colorants, flavorants and glidants that are conventional in the pharmaceutical art.

In order to facilitate the preparation of a solid, controlled-release oral dosage form according to the invention there is provided, in a further aspect of the present invention, a process for the preparation of a solid, controlled-release oral dosage form according to the present invention comprising incorporating the buprenorphine or a salt thereof in a controlled-release matrix. Incorporation in the matrix may be effected, for example, by (a) forming granules comprising at least one hydrophobic and/or hydrophilic material as set forth above (e.g., a water soluble hydroxyalkyl cellulose) together with the buprenorphine; (b) mixing the at least one hydrophobic and/or hydrophilic material-containing granules with at least one C₁₂-C₃₆ aliphatic alcohol, and (c) optionally, compressing and shaping the granules.

The granules may be formed by any of the procedures well-known to those skilled in the art of pharmaceutical formulation. For example, in one preferred method, the granules may be formed by wet granulating hydroxyalkyl cellulose/buprenorphine with water. In a particular preferred embodiment of this process, the amount of water added during the wet granulation step is preferably between 1.5 and 5 times, especially between 1.75 and 3.5 times, the dry weight of the buprenorphine.

In certain embodiments, the dosage form comprises a plurality of matrices described above.

The matrices of the present invention may also be prepared via a melt pellitization technique. In such circumstance, the buprenorphine in finely divided form is combined with a binder (also in particulate form) and other optional inert ingredients, and thereafter the mixture is pelletized, e.g., by mechanically working the mixture in a high shear mixer to form the pellets (granules, spheres). Thereafter, the pellets (granules, spheres) may be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

Controlled-release matrices can also be prepared by, e.g., melt-granulation or melt-extrusion techniques. Generally, melt-granulation techniques involve melting a normally solid hydrophobic binder material, e.g. a wax, and incorporating a powdered drug therein. To obtain a controlled release dosage form, it may be necessary to incorporate a hydrophobic controlled release material, e.g. ethylcellulose or a water-insoluble acrylic polymer, into the molten wax hydrophobic binder material.

The hydrophobic hinder material may comprise one or more water-insoluble wax-like thermoplastic substances possibly mixed with one or more wax-like thermoplastic substances being less hydrophobic than said one or more water-insoluble wax-like substances. In order to achieve controlled release, the individual wax-like substances in the formulation should be substantially non-degradable and insoluble in gastrointestinal fluids during the initial release phases. Useful water-insoluble wax-like binder substances may be those with a water-solubility that is lower than about 1:5,000 (w/w).

In addition to the above ingredients, a controlled release may also contain suitable quantities of other materials, e.g., diluents, lubricants, binders, granulating aids, colorants, flavorants and glidants that are conventional in the pharmaceutical art in amounts up to about 50% by weight of the particulate if desired. The quantities of these additional materials will be sufficient to provide the desired effect to the desired formulation.

The preparation of a suitable melt-extruded matrix according to the present invention may, for example, include the steps of blending the buprenorphine, together with a controlled release material and preferably a binder material to obtain a homogeneous mixture. The homogeneous mixture is then heated to a temperature sufficient to at least soften the mixture sufficiently to extrude the same. The resulting homogeneous mixture is then extruded, using a twin-screw extruder, to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides controlled release of the therapeutically active agent for a time period of from about 6 to at least about 24 hours.

An optional process for preparing the melt extrusioned formulations of the present invention includes directly metering into an extruder a hydrophobic controlled release material, a therapeutically active agent, and an optional binder material; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

Plasticizers, such as those described herein, may be included in melt-extruded matrices. The plasticizer is preferably included as from about 0.1 to about 30% by weight of the matrix. Other pharmaceutical excipients, e.g., talc, mono or poly saccharides, colorants, flavorants, lubricants and the like may be included in the controlled release matrices of the present invention as desired. The amounts included will depend upon the desired characteristic to be achieved.

The diameter of the extruder aperture or exit port can be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic controlled release material as described herein. Preferably the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one preferred embodiment, oral dosage forms are prepared that include an effective amount of melt-extruded multiparticulates within a capsule. For example, a plurality of the melt-extruded multiparticulates may be placed in a gelatin capsule in an amount sufficient to provide an effective controlled release dose when ingested and contacted by gastric

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, 21^{st} ed., 2005 incorporated by reference herein.

In yet another preferred embodiment, the extrudate can be shaped into tablets as set forth in U.S. Pat. No. 4,957,681, hereby incorporated by reference.

Optionally, the controlled-release matrix multiparticulate systems or tablets can be coated, or the gelatin capsule can be further coated, with a controlled release coating such as the controlled release coatings described above. Such coatings preferably include a sufficient amount of hydrophobic and/or hydrophilic controlled-release material to obtain a weight gain level from about 2 to about 25 percent, although the overcoat may be greater depending upon, e.g., the physical properties of the drug and the desired release rate, among other things.

The dosage forms of the present invention may further include combinations of melt-extruded multiparticulates containing one or more drugs. Furthermore, the dosage forms can also include an amount of an immediate release therapeutically active agent for prompt therapeutic effect. The immediate release therapeutically active agent may be incorporated, e.g., as separate pellets within a gelatin capsule, or may be coated on the surface of e.g., melt extruded multiparticulates. The unit dosage forms of the present invention may also contain a combination of, e.g., controlled release beads and matrix multiparticulates to achieve a desired effect.

The controlled-release formulations of the present invention preferably slowly release the therapeutically active agent, e.g., when ingested and exposed to gastric fluids, and then to intestinal fluids. The controlled-release profile of the melt-extruded formulations of the invention can be altered, for example, by varying the amount of controlled-release material, by varying the amount of plasticizer relative to other matrix constituents, hydrophobic material, by the inclusion of additional ingredients or excipients, by altering the method of manufacture, etc.

In other embodiments of the invention, melt-extruded formulations are prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

Typical melt-extrusion production systems suitable for use in accordance with the present invention include a suitable extruder drive motor having variable speed and constant torque control, start-stop controls, and ammeter. In addition, the production system will include a temperature control console which includes temperature sensors, cooling means and temperature indicators throughout the length of the extruder. In addition, the production system will include an extruder such as twin-screw extruder which consists of two counter-rotating intermeshing screws enclosed within a cylinder or barrel having an aperture or die at the exit thereof. The feed materials enter through a feed hopper and are moved through the barrel by the screws and are forced through the die into strands which are thereafter conveyed such as by a continuous movable belt to allow for cooling and being directed to a pelletizer or other suitable device to render the extruded ropes into the multiparticulate system. The pelletizer can consist of rollers, fixed knife, rotating cutter and the like. Suitable instruments and systems will be apparent to those of ordinary skill in the art.

A further aspect of the invention is related to the preparation of melt-extruded multiparticulates as set forth above in a manner which controls the amount of air included in the extruded product. By controlling the amount of air included in the extrudate, the release rate of the therapeutically active agent from the, e.g., multiparticulate extrudate, can be altered significantly. In certain embodiments, the pH dependency of the extruded product can be altered as well.

Thus, in a further aspect of the invention, the melt-extruded product is prepared in a manner which substantially excludes air during the extrusion phase of the process. This may be accomplished, for example, by using a Leistritz extruder having a vacuum attachment. In certain preferred embodiments the extruded multiparticulates prepared according to the invention using the Leistritz extruder under vacuum provides a melt-extruded product having different physical characteristics. In particular, the extrudate is substantially non-porous when magnified, e.g., using a scanning electron microscope which provides an SEM (scanning electron micrograph). Such substantially non-porous formulations provide a faster release of the therapeutically active agent, relative to the same formulation prepared without vacuum. SEMs of the multiparticulates prepared using an extruder under vacuum appear very smooth, and the multiparticulates tend to be more robust than those multiparticulates prepared without vacuum. In certain formulations, the use of extrusion under vacuum provides an extruded multiparticulate product which is more pH-dependent than its counterpart formulation prepared without vacuum. Alternatively, the melt-extruded product is prepared using a Werner-Pfleiderer twin screw extruder.

In certain embodiments, a spheronizing agent is added to a granulate or multiparticulates of the present invention and then spheronized to produce controlled release spheroids. The spheroids are then optionally overcoated with a controlled release coating by methods such as those described herein.

Spheronizing agents which may be used to prepare the multiparticulate formulations of the present invention include any art-known spheronizing agent. Cellulose derivatives are preferred, and microcrystalline cellulose is especially preferred. A suitable microcrystalline cellulose is, for example, the material sold as Avicel™ PH 101. The spheronizing agent is preferably included as about 1 to about 99% of the multiparticulate by weight.

In addition to the active ingredient and spheronizing agent, the spheroids may also contain a binder. Suitable binders, such as low viscosity, water soluble polymers, will be well known to those skilled in the pharmaceutical art. However, water soluble hydroxy lower alkylcellulose, such as hydroxypropylcellulose, are preferred.

In addition to the buprenorphine and spheronizing agent, the multiparticulate formulations of the present invention may include a controlled release material such as those described hereinabove. Preferred controlled-release materials for inclusion in the multiparticulate formulations include acrylic and methacrylic acid polymers or copolymers, and ethylcellulose. When present in the formulation, the controlled-release material will be included in amounts of from about 1 to about 80% of the multiparticulate, by weight. The controlled-release material is preferably included in the multiparticulate formulation in an amount effective to provide controlled release of the buprenorphine from the multiparticulate.

Pharmaceutical processing aids such as binders, diluents, and the like may be included in the multiparticulate formulations. Amounts of these agents included in the formulations will vary with the desired effect to be exhibited by the formulation.

Specific examples of pharmaceutically acceptable carriers and excipients that may be used to formulate oral dosage forms of the present invention are described in the Handbook of Pharmaceutical Excipients, APhA Publications; 5 edition (Jan. 5, 2006) incorporated by reference herein.

The multiparticulates may be overcoated with a controlled-release coating including a controlled-release material such as those described hereinabove. The controlled-release coating is applied to a weight gain of from about 5 to about 30%. The amount of the controlled-release coating to be applied will vary according to a variety of factors, e.g., the composition of the multiparticulate and the chemical and/or physical properties of the drug.

Matrix multiparticulates may also be prepared by granulating the spheronizing agent together with the buprenorphine, e.g. by wet granulation. The granulate is then spheronized to produce the matrix multiparticulates. The matrix multiparticulates are then optionally overcoated with the controlled release coating by methods such as those described hereinabove.

Another method for preparing matrix multiparticulates, for example, by (a) forming granules comprising at least one water soluble hydroxyalkyl cellulose and the buprenorphine or the buprenorphine salt; (b) mixing the hydroxyalkyl cellulose containing granules with at least one C₁₂-C₃₆ aliphatic alcohol; and (c) optionally, compressing and shaping the granules. Preferably, the granules are formed by wet granulating the hydroxyalkyl cellulose/buprenorphine with water. In a particularly preferred embodiment of this process, the amount of water added during the wet granulation step is preferably between 1.5 and 5 times, especially between 1.75 and 3.5 times, the dry weight of the buprenorphine.

In yet other alternative embodiments, a spheronizing agent, together with the active ingredient can be spheronized to form spheroids. Microcrystalline cellulose is preferred. A suitable microcrystalline cellulose is, for example, the material sold as Avicel™ PH 101. In such embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate co-polymer, or ethyl cellulose. In such embodiments, the sustained-release coating will generally include a water insoluble material such as (a) a wax, either alone or in admixture with a fatty alcohol; or (b) shellac or zein.

Spheroids of the present invention comprise a matrix formulation as described above or bead formulation as described hereinafter having a diameter of between 0.1 mm and 2.5 mm, especially between 0.5 mm and 2 mm.

The spheroids are preferably film coated with a controlled release material that permits release of the buprenorphine (or salt) at a controlled rate in an aqueous medium. The film coat is chosen so as to achieve, in combination with the other stated properties, the in-vitro release rate outlined above (e.g., at least about 12.5% released after 1 hour). The controlled-release coating formulations of the present invention preferably produce a strong, continuous film that is smooth and elegant, capable of supporting pigments and other coating additives, non-toxic, inert, and tack-free.

Preparation of Coated Bead Formulations

In certain preferred embodiments of the present invention the oral solid controlled release dosage form of the present invention comprises a plurality of coated substrates, e.g., inert pharmaceutical beads such as nu pariel 18/20 beads. An aqueous dispersion of hydrophobic material is used to coat the beads to provide for the controlled release of the buprenorphine. In certain preferred embodiments a plurality of the resultant stabilized solid controlled-release beads may be placed in a gelatin capsule in an amount sufficient to provide an effective controlled-release dose when ingested and contacted by an environmental fluid, e.g., gastric fluid or dissolution media.

The stabilized controlled-release bead formulations of the present invention slowly release the buprenorphine, e.g., when ingested and exposed to gastric fluids, and then to intestinal fluids. The controlled-release profile of the formulations of the invention can be altered, for example, by varying the amount of overcoating with the aqueous dispersion of hydrophobic controlled release material, altering the manner in which the plasticizer is added to the aqueous dispersion of hydrophobic controlled release material, by varying the amount of plasticizer relative to hydrophobic controlled release material, by the inclusion of additional ingredients or excipients, by altering the method of manufacture, etc. The dissolution profile of the ultimate product may also be modified, for example, by increasing or decreasing the thickness of the controlled release coating.

Substrates coated with a therapeutically active agent are prepared, e.g. by dissolving the therapeutically active agent in water and then spraying the solution onto a substrate, for example, nu pariel 18/20 beads, using a Wuster insert. Optionally, additional ingredients are also added prior to coating the beads in order to assist the binding of the buprenorphine to the beads, and/or to color the solution, etc. For example, a product which includes hydroxypropyl methylcellulose, etc. with or without colorant (e.g., Opadry™) may be added to the solution and the solution mixed (e.g., for about 1 hour) prior to application of the same onto the substrate. The resultant coated substrate may then be optionally overcoated with a barrier agent, to separate the therapeutically active agent from the hydrophobic controlled-release coating.

An example of a suitable barrier agent is one which comprises hydroxypropyl methylcellulose. However, any film-former known in the art may be used. It is preferred that the barrier agent does not affect the dissolution rate of the final product.

The substrates may then be overcoated with an aqueous dispersion of the hydrophobic controlled release material as described herein. The aqueous dispersion of hydrophobic controlled release material preferably further includes an effective amount of plasticizer, e.g. tri-ethyl citrate. Pre-formulated aqueous dispersions of ethylcellulose, such as Aquacoat™ or Surelease™, may be used. If Surelease™ is used, it is not necessary to separately add a plasticizer. Alternatively, pre-formulated aqueous dispersions of acrylic polymers such as Eudragit™ can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color can be added to Aquacoat™ via the use of alcohol or propylene glycol based color dispersions, milled aluminum lakes and opacifiers such as titanium dioxide by adding color with shear to water soluble polymer solution and then using low shear to the plasticized Aquacoat™. Alternatively, any suitable method of providing color to dioxide and color pigments, such as iron oxide pigments. The incorporation of pigments, may, however, increase the retard effect of the coating.

The plasticized aqueous dispersion of hydrophobic controlled release material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined controlled-release of said therapeutically active agent when said coated substrate is exposed to aqueous solutions, e.g. gastric fluid, is preferably applied, taking into account the physical characteristics of the therapeutically active agent, the manner of incorporation of the plasticizer, etc. After coating with the hydrophobic controlled release material, a further overcoat of a film-former, such as Opadry™, is optionally applied to the beads. This overcoat is provided, if at all, in order to substantially reduce agglomeration of the beads.

Another method of producing controlled release bead formulations suitable for about 24-hour administration is via powder layering. The powder-layered beads are prepared by spraying an aqueous binder solution onto inert beads to provide a tacky surface, and subsequently spraying a powder that is a homogenous mixture of the buprenorphine and hydrous lactose impalpable onto the tacky beads. The beads are then dried and coated with a hydrophobic material such as those described hereinabove to obtain the desired release of drug when the final formulation is exposed to environmental fluids. An appropriate amount of the controlled release beads are then, e.g. encapsulated to provide a final dosage form which provides effective plasma concentrations for the intended duration of effect or dosing frequency.

Controlled Release Osmotic Dosage

Controlled release dosage forms according to the present invent on may also be prepared as osmotic dosage formulations. The osmotic dosage forms preferably include a bilayer core comprising a drug layer and a delivery or push layer, wherein the bilayer core is surrounded by a semipermeable wall and optionally having at least one passageway disposed therein. In certain embodiments, the bilayer core comprises a drug layer with the buprenorphine or a salt thereof and a displacement or push layer. In certain preferred embodiments the drug layer may also comprise at least one polymer hydrogel. The polymer hydrogel may have an average molecular weight of between about 500 and about 6,000,000. Examples of polymer hydrogels include but are not limited to a maltodextrin polymer comprising the formula (C₆H₁₂O₅). H2O, wherein n is 3 to 7,500, and the maltodextrin polymer comprises a 500 to 1,250,000 number-average molecular weight; a poly(alkylene oxide) represented by, e.g., a poly(ethylene oxide) and a poly(propylene oxide) having a 50,000 to 750,000 weight-average molecular weight, and more specifically represented by a poly(ethylene oxide) of at least one of 100,000, 200,000, 300,000 or 400,000 weight-average molecular weights; an alkali carboxyalkylcellulose, wherein the alkali is sodium or potassium, the alkyl is methyl, ethyl, propyl, or butyl of 10,000 to 175,000 weight-average molecular weight; and a copolymer of ethylene-acrylic acid, including methacrylic and ethacrylic acid of 10,000 to 500,000 number-average molecular weight.

In certain preferred embodiments of the present invention, the delivery or push layer comprises an osmopolymer. Examples of an osmopolymer include but are not limited to a member selected from the group consisting of a polyalkylene oxide and a carboxyalkylcellulose. The polyalkylene oxide possesses a 1,000,000 to 10,000,000 weight-average molecular weight. The polyalkylene oxide may be a member selected from the group consisting of polymethylene oxide, polyethylene oxide, polypropylene oxide, polyethylene oxide having a 1,000,000 average molecular weight, polyethylene oxide comprising a 5,000,000 average molecular weight, polyethylene oxide comprising a 7,000,000 average molecular weight, cross-linked polymethylene oxide possessing a 1,000,000 average molecular weight, and polypropylene oxide of 1,200,000 average molecular weight. Typical osmopolymer carboxyalkylcellulose comprises a member selected from the group consisting of alkali carboxyalkylcellulose, sodium carboxymethylcellulose, potassium carboxymethylcellulose, sodium carboxyethylcellulose, lithium carboxymethylcellulose, sodium carboxyethylcellulose, carboxyalkylhydroxyalkylcellulose, carboxymethylhydroxyethyl cellulose, carboxyethylhydroxyethylcellulose and carboxymethylhydroxypropylcellulose. The osmopolymers used for the displacement layer exhibit an osmotic pressure gradient across the semipermeable wall. The osmopolymers imbibe fluid into dosage form, thereby swelling and expanding as an osmotic hydrogel (also known as osmogel), whereby they push the buprenorphine or pharmaceutically acceptable salt thereof from the osmotic dosage form.

The push layer may also include one or more osmotically effective compounds also known as osmagents and as osmotically effective solutes. They imbibe an environmental fluid, for example, from the gastrointestinal tract, into dosage form and contribute to the delivery kinetics of the displacement layer. Examples of osmotically active compounds comprise a member selected from the group consisting of osmotic salts and osmotic carbohydrates. Examples of specific osmagents include but are not limited to sodium chloride, potassium chloride, magnesium sulfate, lithium phosphate, lithium chloride, sodium phosphate, potassium sulfate, sodium sulfate, potassium phosphate, glucose, fructose and maltose.

The push layer may optionally include a hydroxypropylalkylcellulose represented by a member selected from the group consisting of hydroxypropylmethylcellulose, hydroxypropylethylcellulose, hydroxypropyl isopropylcellulose, hydroxypropylbutylcellulose, and hydroxypropyl pentylcellulose.

The push layer optionally may comprise a nontoxic colorant or dye. Examples of colorants or dyes include but are not limited to Food and Drug Administration Colorant (FD&C), such as FD&C No. 1 blue dye, FD&C No. 4 red dye, red ferric oxide, yellow ferric oxide, titanium dioxide, carbon black, and indigo.

The push layer may also optionally comprise an antioxidant to inhibit the oxidation of ingredients. Some examples of antioxidants include but are not limited to a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate.

In certain alternative embodiments, the dosage form comprises an homogenous core comprising the buprenorphine or a pharmaceutically acceptable salt thereof, a pharmaceutically acceptable polymer (e.g., polyethylene oxide), optionally a disintegrant (e.g., polyvinylpyrrolidone), optionally an absorption enhancer (e.g., a fatty acid, a surfactant, a cheating agent, a bile salt, etc.). The homogenous core is surrounded by a semipermeable wall having a passageway (as defined above) for the release of the buprenorphine or pharmaceutically acceptable salt thereof.

In certain embodiments, the semipermeable wall comprises a member selected from the group consisting of a cellulose ester polymer, a cellulose ether polymer and a cellulose ester-ether polymer. Representative wall polymers comprise a member selected from the group consisting of cellulose acylate, cellulose diacylate, cellulose triacylate, cellulose acetate, cellulose diacetate, cellulose triacetate, mono-, di- and tricellutose alkenylates, and mono-, di- and tricellutose alkinylates. The poly(cellulose) used for the present invention comprises a number-average molecular weight of 20,000 to 7,500,000.

Additional semipermeable polymers for the purpose of this invention comprise acetaldehyde dimethycellulose acetate, cellulose acetate ethylcarbamate, cellulose acetate methylcarbamate, cellulose diacetate, propylcarbamate, cellulose acetate diethylaminoacetate; semipermeable polyamide; semipermeable polyurethane; semipermeable sulfonated polystyrene; semipermeable cross-linked polymer formed by the coprecipitation of a polyanion and a polycation as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable crosslinked polystyrenes; semipermeable cross-linked poly(sodium styrene sulfonate); semipermeable crosslinked poly(vinylbenzyltrimethyl ammonium chloride); and semipermeable polymers possessing a fluid permeability of 2.5×10⁻⁸ to 2.5×10⁻² (cm²/hr·atm) expressed per atmosphere of hydrostatic or osmotic pressure difference across the semipermeable wall. Other polymers useful in the present invention are known in the art in U.S. Pat. Nos. 3,845;770; 3,916,899 and 4,160,020; and in Handbook of Common Polymers, Scott, J. R. and W. J. Roff, 1971, CRC Press, Cleveland, Ohio.

In certain embodiments, preferably the semipermeable wall is nontoxic, inert, and it maintains its physical and chemical integrity during the dispensing life of the drug. In certain embodiments, the dosage form comprises a binder as described above.

In certain embodiments, the dosage form comprises a lubricant, which may be used during the manufacture of the dosage form to prevent sticking to die wall or punch faces. Examples of lubricants include but are not limited to magnesium stearate, sodium stearate, stearic acid, calcium stearate, magnesium oleate, oleic acid, potassium oleate, caprylic acid, sodium stearyl fumarate, and magnesium palmitate.

Coatings

The dosage forms of the present invention may optionally be coated with one or more coatings suitable for the regulation of release or for the protection of the formulation. In one embodiment, coatings are provided to permit either pH-dependent or pH-independent release, e.g., when exposed to gastrointestinal fluid. When a pH-independent coating is desired, the coating is designed to achieve optimal release regardless of pH-changes in the environmental fluid, e.g., the GI tract. Other preferred embodiments include a pH-dependent coating that releases the buprenorphine in desired areas of the gastrointestinal (GI) tract, e.g., the stomach or small intestine, such that an absorption profile is provided which is capable of providing at least about twelve hour and preferably up to twenty-four hour analgesia to a patient. It is also possible to formulate compositions which release a portion of the dose in one desired area of the GI tract, e.g., the stomach, and release the remainder of the dose in another area of the GI tract, the small intestine.

Formulations according to the invention that utilize pH-dependent coatings my also impart a repeat-action effect whereby unprotected drug is coated over an enteric coat and is released in the stomach, while the remainder, being protected by the enteric coating, is released further down the gastrointestinal tract. Coatings which are pH-dependent may be used in accordance with the present invention include a controlled release material such as, e.g., shellac, cellulose acetate phthalate (CAP), polyvinyl acetate phthalate (PVAP), hydroxypropyl methylcellulose phthalate, and methacrylic acid ester copolymers, zein, and the like.

In another preferred embodiment, the present invention is related to a stabilized solid controlled dosage form comprising the buprenorphine coated with a hydrophobic controlled release material selected from (i) an alkylcellulose; (ii) an acrylic polymer; or (iii) mixtures thereof. The coating may be applied in the form of an organic or aqueous solution or dispersion.

In certain preferred embodiments, the controlled release coating is derived from an aqueous dispersion of the hydrophobic controlled release material. The coated substrate containing the buprenorphine (e.g., a tablet core or inert pharmaceutical beads or spheroids) is then cured until an endpoint is reached at which the substrate provides a stable dissolution. The curing endpoint may be determined by comparing the dissolution profile (curve) of the dosage form immediately after curing to the dissolution profile (curve) of the dosage form after exposure to accelerated storage conditions of e.g., at least one month at a temperature of 40° C. and a relative humidity of 75%.

In preferred embodiments, the controlled release coatings include a plasticizer such as those described herein.

In certain embodiments, it is necessary to overcoat the substrate comprising the buprenorphine with a sufficient amount of the aqueous dispersion of e.g., alkylcellulose or acrylic polymer, to obtain a weight gain level from about 2 to about 50%, e.g., about 2 to about 25% in order to obtain a controlled-release formulation. The overcoat may be lesser or greater depending upon the physical properties of the therapeutically active agent and the desired release rate, the inclusion of plasticizer in the aqueous dispersion and the manner of incorporation of the same, for example.

Alkylcellulose Polymers

Cellulosic materials and polymers, including alkylcelluloses are controlled release materials well suited for coating the substrates, e.g., beads, tablets, etc. according to the invention. Simply by way of example, one preferred alkylcellulosic polymer is ethylcellulose, although the artisan will appreciate that other cellulose and/or alkylcellulose polymers may be readily employed, singly or on any combination, as all or part of a hydrophobic coating according to the invention.

One commercially-available aqueous dispersion of ethylcellulose is Aquacoat™. Aquacoat™ is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudotatex during the manufacturing phase. Thus, prior to using the same as a coating, it is necessary to intimately mix the Aquacoat™ with a suitable plasticizer prior to use.

Another aqueous dispersion of ethylcellulose is commercially available as Surelease™. This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Acrylic Polymers

In other preferred embodiments of the present invention, the controlled release material comprising the controlled-release coating is a pharmaceutically acceptable acrylic polymer, including but not limited to acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit™. There are several different types of Eudragit™. For example, Eudragit™ E is an example of a methacrylic acid copolymer which swells and dissolves in acidic media. Eudragit™ L is a methacrylic acid copolymer which does not swell at about pH<5.7 and is soluble at about pH>6. Eudragit™ S does not swell at about pH<6.5 and is soluble at about pH>7. Eudragit™ RL and Eudragit™ RS are water swellable, and the amount of water absorbed by these polymers is pH-dependent, however, dosage forms coated with Eudragit™ RL and RS are pH-independent.

In certain preferred embodiments, the acrylic coating comprises a mixture of two acrylic resin lacquers commercially available as Eudragit™ RL30D and Eudragit™ RS30D, respectively. Eudragit™ RL30D and Eudragit™ RS30D are copolymers of acrylic and methacrylic esters with a low content of quaternary ammonium groups, the molar ratio of ammonium groups to the remaining neutral (meth)acrylic esters being 1:20 in Eudragit™ RL30D and 1:40 in Eudragit™ RS30D. The mean molecular weight is about 150,000. The code designations RL (high permeability) and RS (low permeability) refer to the permeability properties of these agents. Eudragit™ RL/RS mixtures are insoluble in water and in digestive fluids. However, coatings formed from the same are swellable and permeable in aqueous solutions and digestive fluids.

The Eudragit™ RL/RS dispersions of the present invention may be mixed together in any desired ratio in order to ultimately obtain a controlled-release formulation having a desirable dissolution profile. Desirable controlled-release formulations may he obtained, for instance, from a retardant coating derived from 100% Eudragit™ RL, 50% Eudragit™ RL and 50% Eudragit™ RS, and 10% Eudragit™ RL:Eudragit™ 90% RS. Of course, one skilled in the art will recognize that other acrylic polymers may also be used, such as, for example, Eudraget™ L.

Plasticizers

In embodiments of the present invention where the coating comprises an aqueous dispersion of a hydrophobic controlled release material, the inclusion of an effective amount of a plasticizer in the aqueous dispersion of hydrophobic material will further improve the physical properties of the controlled-release coating. For example, because ethylcellulose has a relatively high glass transition temperature and does not form flexible films under normal coating conditions, it is preferable to incorporate a plasticizer into an ethylcellulose coating containing controlled-release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tibutyl citrate, and triacetin, although it is possible that other water-insoluble plasticizers such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit™ RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

In certain embodiments, the addition of a small amount of talc to the controlled release coating reduces the tendency of the aqueous dispersion to stick during processing, and acts as a polishing agent.

The release of the therapeutically active agent from the controlled-release formulation of the present invention can be further influenced, i.e., adjusted to a desired rate, by the addition of one or more release-modifying agents, or by providing one or more passageways through the coating. The ratio of hydrophobic controlled release material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The controlled-release coatings of the present invention can also include erosion-promoting agents such as starch and gums.

The controlled-release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer. In certain preferred embodiments, the release-modifying agent is selected from hydroxypropylmethylcellulose, lactose, metal stearates, and mixtures of any of the foregoing.

The controlled-release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864, all of which are hereby incorporated by reference. The passageway can have any shape such as round, triangular, square, elliptical, irregular, etc.

The following examples illustrate various aspects of the present invention. They are not to be construed to limit the claims in any manner whatsoever. A wide variety of methods known in the art for the preparation of oral immediate release and oral controlled release dosage forms may be incorporated into the invention. Other suitable dosage forms may also be prepared by modification of the examples herein and by use of material other than those specifically disclosed herein, including those which may hereafter become known to the art to be capable of performing the necessary functions. Other suitable modifications and adaptations of the variety of conditions and parameters normally encountered and obvious to those skilled in the art are within the spirit and scope of the invention.

The percent loading of the buprenorphine onto the dosage form may be varied depending on the physiochemical and pharmaceutical properties of immediate release and controlled release material, excipients, the selected salt of buprenorphine and the desired release profile and duration of actions.

The ingredients used for the preparation of the buprenorphine dosage form agent may be modified depending on the selection, dose and desired duration of effect. In some embodiments, a change in the dose or amount buprenorphine will not require a significant change in amount of other ingredients. In other embodiments, a proportional change in the amount of other ingredients is required to maintain the desired properties. In yet other embodiments, a change in the dose or amount buprenorphine necessitates a change in the nature and/or amount of ingredients to provide the required characteristics of the buprenorphine duration of effect, rate and extent of absorption, therapeutic concentrations and effect, etc.).

### EXAMPLE 1

Tablet Composition of Extended Release Buprenorphine HCl

| Ingredients | Qty./Unit |
|---|---|
| 1. Buprenorphine HCl | 20 mg |
| 2. HPMC 2208, USP | 150 mg |
| 3. Carnauba wax | 30 mg |
| 4. HPMC 2910, USP | 15 mg |
| 5. Magnesium Stearate | 2 mg |
| 6. Stearic acid | 8 mg |
| 7. Talc | 3 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### EXAMPLE 2

Tablet Composition of Extended Release Buprenorphine HCl

| Ingredients | Qty./Unit |
|---|---|
| 1. Buprenorphine HCl | 100 mg |
| 2. HPMC 2208, USP | 250 mg |
| 3. Carnauba wax | 50 mg |
| 4. HPMC 2910, USP | 25 mg |
| 5. Magnesium Stearate | 4 mg |
| 6. Stearic acid | 14 mg |
| 7. Talc | 5 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### EXAMPLE 3

Tablet Composition of Extended Release Buprenorphine HCl

| Ingredients | Qty./Unit |
|---|---|
| 1. Buprenorphine HCl | 500 mg |
| 2. HPMC 2208, USP | 250 mg |
| 3. Carnauba wax | 50 mg |
| 4. HPMC 2910, USP | 25 mg |
| 5. Magnesium Stearate | 4 mg |
| 6. Stearic acid | 14 mg |
| 7. Talc | 5 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### EXAMPLE 4

Tablet Composition of Extended Release Buprenorphine Base

| Ingredients | Amt/Unit (mg) |
|---|---|
| Buprenorphine Base | 20 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### EXAMPLE 5

Tablet Composition of Extended Release Buprenorphine HCl

| Ingredients | Amt/Unit (mg) |
|---|---|
| Buprenorphine HCl | 30 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### EXAMPLE 6

Capsule Composition of Extended Release Buprenorphine Base

| Ingredients | Amt/Unit (mg) |
|---|---|
| Buprenorphine Base | 20 |
| Eudragit RSPO | 76 |
| Eudragit RLPO | 4 |
| Stearyl Alcohol | 25 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum-about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### EXAMPLE 7

Capsule Composition of Extended Release Buprenorphine Base

| Ingredients | Amt/Unit (mg) |
|---|---|
| Buprenorphine Base | 200 |
| Eudragit RSPO | 150 |
| Eudragit RLPO | 10 |
| Stearyl Alcohol | 40 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum-about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### EXAMPLE 8

Capsule Composition of Extended Release Buprenorphine Base

| Ingredients | Amt/Unit (mg) |
|---|---|
| Buprenorphine Base | 15 |
| Eudragit RSPO | 77 |
| Ethocel | 4.5 |
| Stearic acid | 27 |

Blend milled Stearic acid, Ethocel, Buprenorphine Base, and Eudragit RSPO using a V-blender. 2. Extrude the mixture using a Powder Feeder, Melt Extruder (equipped with the 6×1 mm die head), Conveyor, Lasermike, and Pelletizer. Powder feed rate, 1.2 kg/hr; vacuum, about 980 mBar; Conveyor, such that diameter of extrudate is 1 mm; Pelletizer, such that pellets are cut to 1 mm in length. 3. Screen pellets using #16 mesh and #20 mesh screens. Collect material that passes through the #16 mesh screen and is retained on the #20 mesh screen. Fill pellets in capsules.

### EXAMPLE 9

Capsule Composition of Extended Release Buprenorphine HCl

| Steps | Ingredients | Amt/unit (mg) |
|---|---|---|
| 1 | Buprenorphine HCl | 12 |
| | Non-pareil beads (30/35 mesh) | 45 |
| | Opadry Clear | 2.5 |
| 2 | Eudragit RS3-D (dry) | 7.2 |
| | Eudragit RL30D (dry) | 0.4 |
| | Triethyl citrate | 1.5 |
| | Cabosil | 0.4 |
| 3 | Opadry Clear (HPMC) | 1.9 |
| | Cabosil | 0.28 |

1. Dissolve Buprenorphine HCl and Opadry (HPMC) in water. Spray the drug solution onto nonpareil beads in a fluid bed coater with Wurster insert. 2, Disperse Eudragit RS, Eudragit RL, triethyl citrate, and Cabosil in water. Spray the dispersion onto the beads in the fluid bed coater. 3. Dissolve Opadry in water. Spray the solution onto the beads in the fluid bed coater. 4. Cure the beads at 60.degree. C. for 24 hours.

### EXAMPLE 10

Tablet Composition of Extended Release Buprenorphine HCl

| Ingredient | Amt/unit (mg) |
|---|---|
| Buprenorphine HCl | 75 |
| Anhydrous Dicalcium | 60 |
| Phosphate (Powdered) | 80 |
| Microcrystalline Cellulose | 80 |
| Glyceryl Behenate | 40 |
| Magnesium Stearate | 4 |
| Opadry Red | 17 |
| Purified Water | 900* |

*Remains in product as residual moisture only.

1. Pass the Stearyl Alcohol flakes through an oscillating mill. 2. Mix the Buprenorphine HCl, milled Stearyl Alcohol, Anhydrous Dicalcium Phosphate, Microcrystalline Cellulose, and Glyceryl Behenate in a twin shell blender. 3. Continuously feed the blended material into a twin screw extruder and collect the resultant heated material on a conveyor. 4. Allow the extrudate to cool on the conveyor. 5. Mill the cooled extrudate using an oscillating mill. 6. Blend the milled extrudate and Magnesium Stearate, 7. Compress the resultant granulation using a tablet press, preferably into a caplet. 8. Prepare a film coating solution by dispersing the Opadry in Purified Water and applying it to the tablet.

### EXAMPLE 11

Capsule Composition of Extended Release Buprenorphine HCl

| Ingredient | Amt/unit (mg) |
|---|---|
| Buprenorphine HCl | 20 |
| Eudragit RSPO | 76.5 |
| Ethylcellulose | 4.5 |
| Stearyl Alcohol | 27 |

1. Pass Stearyl Alcohol flakes through an impact mill. 2. Mix the Buprenorphine HCl, Eudragit, Ethylcellulose and milled Stearyl Alcohol in a twin shell blender. 3. Continuously feed the blended material into a twin screw extruder and collect the resultant strands on a conveyor. 4. Allow the strands to cool on the conveyor. 5. Cut the cooled strands into pellets using a Pelletizer. 6. Screen the pellets and collect desired sieve portion. 7. Fill the extruded pellets into capsules.

Example 12 to 23 may be prepared as follows: (i) Dispense the specified hydrophobic controlled release material (e.g., hydrogenated Type I vegetable oil, hydrogenated Type II vegetable oil, polyoxyethylene stearates, polyoxyethylene distearates, glycerol monostearate, poorly water soluble, or high melting point waxes) into a mixer; (ii) Heat until fully incited; (iii) dispense the hydroxypropyl methyl cellulose (HPMC) into the mixer; (iv) Mix until dispersed; (v) Dispense the Aerosil into the same vessel; (vi) Mix until dispersed; (vii) Dispense the buprenorphine into the same vessel; (viii) Stir thoroughly with a high shear mixer; (ix) Transfer the mix into a liquid filling machine; (x) Fill into hard gelatin (or HPMC) capsule; (xi) Optionally, transfer the capsules to a banding machine and band the capsules.

### EXAMPLE 12

Capsule Composition of Extended Release Buprenorphine HCl

| Ingredients | Quantity (mg)/Dose |
|---|---|
| Sterotex ® NF | 200 |
| Fractionated coconut oil | 70 |
| Methocel ® K 15M | 81 |
| Aerosil ® COK 84 | 4 |
| Buprenorphine HCl | 50 |

### EXAMPLE 13

Capsule Composition of Extende Release Buprenorphine HCl

| Ingredients | Quantity (mg)/Dose |
|---|---|
| Beeswax | 200 |
| HPMC, K15M | 80 |
| Aerosil COK 84 | 8 |
| Buprenorphine HCl | 40 |

### EXAMPLE 14

Capsule Composition of Extended Release Buprenorphine Base

| Ingredients | Quantity (mg)/Dose |
|---|---|
| Sterotex NF | 150 |
| HPMC, K15M | 75 |
| Coconut oil | 75 |
| Aerosil COK 84 | 5 |
| Buprenorphine | 100 |

### EXAMPLE 15

Capsule Composition of Extended Release Buprenorphine Base

| Ingredients | Quantity (mg)/Dose |
|---|---|
| Cithrol GMS | 275 |
| HPMC, K100M | 40 |
| Aerosil COK 84 | 10 |
| Buprenorphine | 75 |

### EXAMPLE 16

Capsule Composition of Extended Release Buprenorphine HCl

| Ingredients | Quantity (mg)/Dose |
|---|---|
| Hydrokote 112 | 250 |
| HPMC, K15M | 60 |
| Aerosil COK 84 | 10 |
| Buprenorphine HCl | 150 |

### EXAMPLE 17

Capsule Composition of Extended Release Buprenorphine Base

| Ingredients | Quantity (mg)/Dose |
|---|---|
| Beeswax | 200 |
| HPMC, Pharmacoat | 62.5 |
| 606 | 7.5 |
| Aerosil COK 84 | 200 |
| Buprenorphine Base | |

### EXAMPLE 18

Capsule Composition of Extended Release Buprenorphine Base

| Ingredients | Quantity (mg)/Dose |
|---|---|
| Gelucire 50/02 | 190 |
| Methocel K 100M | 35 |
| Aerosil COK 84 | 10 |
| Buprenorphine Base | 250 |

### EXAMPLE 19

Capsule Composition of Extended Release Buprenorphine Base

| Ingredients | Quantity (mg)/Dose |
|---|---|
| Cetyl alcohol | 280 |
| Methocel K 100M | 50 |
| Aerosil COK 84 | 10 |
| Buprenorphine Base | 100 |

### EXAMPLE 20

Capsule Composition of Extended Release Buprenorphine

| Ingredients | Quantity (mg)/Dose |
|---|---|
| Sterotex NF | 320 |
| Methocel K 15M | 60 |
| Aerosil COK 84 | 10 |
| Buprenorphine HCl | 300 |

### EXAMPLE 21

Capsule Composition of Extended Release Buprenorphine HCl

| Ingredients | Quantity (mg)/Dose |
|---|---|
| Cithrol GMS | 320 |
| Methocel K 100M | 55 |
| Aerosil COK 84 | 15 |
| Buprenorphine HCl | 150 |

### EXAMPLE 22

Capsule Composition of Extended Release Buprenorphine HCl

| Ingredients | Quantity (mg)/Dose |
|---|---|
| Sterotex ® NF | 100 |
| Fractionated coconut oil | 70 |
| Beeswax | 100 |
| Methocel ® K 15M | 81 |
| Aerosil ® COK 84 | 4 |
| Buprenorphine HCl | 200 |

### EXAMPLE 23

Capsule Composition of Extended Release Buprenorphine HCl

| Ingredients | Quantity (mg)/Dose |
|---|---|
| Glyceryl behenate | 135 |
| Fractionated coconut oil | 50 |
| Methocel ® K 15M | 60 |
| Aerosil ® COK 84 | 3 |
| Buprenorphine HCl | 50 |

### EXAMPLE 24

Tablet Composition of Extended Release Buprenorphine HCl

| Ingredients | Quantity (mg)/Dose |
|---|---|
| Lactose (spray dried) | 230 |
| Eudragit ™ RS PM | 60 |
| Purified water | q.s.* |
| Stearyl Alcohol | 90 |
| Talc | 8 |
| Magnesium Stearate | 4 |
| Buprenorphine HCl | 300 |

*Remains in product as residual moisture only.

*Remains in product as residual moisture only.

In Example 24, the required quantities of buprenorphine HCl, spray-dried lactose, and Eudragit198 RS PM are transferred into an appropriate-size mixer, and mixed for approximately 5 minutes. While the powders are mixing, the mixture is granulated with enough water to produce a moist granular mass. The granules are then dried in a fluid bed dryer at 60° C., and then passed through an 8-mesh screen. Thereafter, the granules are re-dried and pushed through a 12-mesh screen. The required quantity of stearyl alcohol is melted at approximately 60 to 70° C., and while the granules are mixing, the melted stearyl alcohol is added. The warm granules are returned to the mixer. The coated granules are removed from the mixer and allowed to cool. The granules are then passed through a 12-mesh screen. The granulate is then lubricated by mixing the required quantity of talc and magnesium stearate in a suitable blender. Tablets are compressed on a suitable tableting machine.

In some embodiments, oral immediate release compressed tablets of buprenorphine can be formulated using conventional wet granulation procedures and equipment.

### EXAMPLE 25

Immediate Release Tablet Composition of Buprenorphine HCl

| Ingredients | Qty./Unit | |
|---|---|---|
| 1. Buprenorphine HCl | 100 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### EXAMPLE 26

Immediate Release Tablet Composition of Buprenorphine HCl

| Ingredients | Qty./Unit | |
|---|---|---|
| 1. Buprenorphine HCl | 50 | mg |
| 2. Polyvinypyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### EXAMPLE 27

Immediate Release Table Composition of Buprenorphine HCl

| Ingredients | Qty./Unit | |
|---|---|---|
| 1. Buprenorphine HCl | 75 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

In Example 25 to 27, blend 1, 2 and 3 together; pass through a 40-mesh screen. Add 4 slowly and knead well. Screen wet mass through a 4-mesh screen. Dry the granulation at 50° C. overnight, Screen the dried granulation through a 20-mesh screen. Bolt 5, 6 and 7 through a 60-mesh screen prior to mixing by tumbling with granulation. Compress using a concave punch.

In some embodiments, oral immediate release compressed tablets of buprenorphine can be formulated using conventional dry granulation procedures and equipment.

### EXAMPLE 28

Tablet Composition of Buprenorphine HCl

| Ingredients | Qty./Unit |
|---|---|
| 1 Buprenorphine HCl | 20 mg |
| 2 Lactose (granular, 12-mesh) | 25 mg |
| 3 Starch | 20 mg |
| 4 Talc | 20 mg |
| 5 Magnesium Stearate | 0.3 mg |

In Example 28, mix ingredients 1 to 5 thoroughly. Compress into slugs. Grind and screen to 14- to 16-mesh granules. Recompress into tablets using a concave punch.

In some embodiments, oral sustained release compressed tablets of buprenorphine can be formulated using conventional fluid-bed granulation procedures and equipment.

### EXAMPLE 29

Tablet Composition of Buprenorphine HCl

| Ingredients | Qty./Unit |
|---|---|
| 1. Buprenorphine HCl | 50 mg |
| 2. HPMC 2208, USP | 150 mg |
| 3. Carnauba wax | 30 mg |
| 4. HPMC 2910, USP | 15 mg |
| 5. Magnesium Stearate | 2 mg |
| 6. Stearic acid | 8 mg |
| 7. Talc | 3 mg |

In Example 29, place the ingredients 1, 2 and 3 in the granulator and mix for 15 minutes. Dissolve ingredient 4 in water (mix in how water, then cool down) and spay into the fluidized mixture. Dry to approximately 5% moisture. Sequentially add ingredient 5, 6 and 7, with mixing steps between each addition. Compress using capsule shaped tooling.

These and other embodiments of the present invention will readily occur to those of ordinary skill in the art in view of the disclosure herein.

Having now fully described the invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

1-167. (canceled)

168. An oral dosage form comprising: (i) a therapeutically effective amount of buprenorphine, a pharmaceutically acceptable salt thereof, or a mixture of these, and (ii) controlled release material to render said dosage form suitable for modified release, said dosage form providing delayed onset of buprenorphine release.

169. The oral dosage form of claim 168, being abuse resistant compared with oral dosage forms of buprenorphine suitable for modified release or extended release or controlled release but without delayed release.

170. The dosage form of claim 169, wherein the dose of said modified release dosage form with delayed onset is at least about 10% greater than the comparator dose.

171. The dosage form of claim 168, wherein the modified release dosage form is a delayed onset, rapid release dosage form.

172. The dosage form of claim 168, wherein the modified release dosage form is a delayed onset, pulsatile release dosage form.

173. The dosage form of claim 168, wherein the modified release dosage form is a delayed onset, extended release dosage form.

174. The oral dosage form of claim 168, providing first release of buprenorphine from the dosage form at a point distal to the stomach.

175. The oral dosage form of claim 168, after a single administration providing first release of buprenorphine from the dosage form not less than about 2 hours after oral ingestion.

176. The oral dosage form of claim 168, after a single administration providing first release of buprenorphine from the dosage form at a pH of about ≧5.

177. The oral dosage form of claim 168, upon initiating release providing delayed onset, rapid release of buprenorphine for up to about 3 hours.

178. The oral dosage form of claim 168, said upon initiating release providing delayed onset, extended release of buprenorphine for up to about 24 hours.

179. The oral dosage form of claim 168, providing an in-vitro buprenorphine release rate, when measured by the USP Basket or Paddle Method at 100 rpm in 900 mL of distilled water at 37° C. which is substantially pH dependent in that a difference, at 2 hours between the amount of buprenorphine released at a pH of ≦5, and an amount released at a pH of ≧6 is more than about 25%.

180. An oral dosage form comprising a therapeutically effective amount of buprenorphine, a pharmaceutically acceptable salt thereof, or a mixture of these, and naloxone, a pharmaceutically acceptable salt thereof, or a mixture thereof, said dosage form having amass ratio of naloxone base relative to buprenorphine base which is at least 50% less than the mass ratio of naloxone base relative to buprenorphine base contained in the commercially marketed sublingual preparation containing buprenorphine and naloxone.

181. The oral dosage form of claim 180, further comprising controlled release material to render said dosage form suitable for modified release.

182. An oral modified release pharmaceutical composition comprising a therapeutically effective amount of buprenorphine, a pharmaceutically acceptable salt thereof or a mixture of these; and a controlled release material to render said dosage form suitable for extended release, or delayed onset extended release in a mammal.
